(19) United States
(12) Patent Application Publication
(10) Pub. No.: US 2020/0258616 A1
(43) Pub. Date: Aug. 13, 2020

(54) AUTOMATED IDENTIFICATION AND GRADING OF INTRAOPERATIVE QUALITY

(71) Applicants: THE REGENTS OF THE UNIVERSITY OF MICHIGAN, Ann Arbor, MI (US); The Brigham and Women's Hospital, Inc., Boston, MA (US)

(72) Inventors: Donald Likosky, Ann Arbor, MI (US); Steven Yule, Boston, MA (US); Francis D. Pagani, South Lyon, MI (US); Michael R. Mathias, Ann Arbor, MI (US); Jason J. Corso, Chelsea, MI (US); Roger Daglius Dias, Cambridge, MA (US); Emily Mower Provost, Ann Arbor, MI (US)

(21) Appl. No.: 16/705,371

(22) Filed: Dec. 6, 2019

Related U.S. Application Data
(60) Provisional application No. 62/802,293, filed on Feb. 7, 2019.

(51) Int. Cl. G16H 40/20; G16H 10/60; H04N 21/44; H04N 21/439; G06N 3/08; G06N 20/20; G06N 20/10

(52) U.S. Cl. CPC G16H 40/20; G16H 10/60; H04N 21/44; G06N 20/10; G06N 3/084; G06N 20/20; H04N 21/439

(57) ABSTRACT

Embodiments described herein relate, inter alia, to receiving one or more segments of a digital recording, wherein the one or segments include video and/or audio data of a surgical procedure; analyzing, via a video/audio understanding model, the one or more segments to (i) characterize a plurality of independent features associated with a technical skill and/or a non-technical practice that are evident in the one or more segments and (ii) determine a higher-order pattern based upon analyzing a group of at least two of the plurality of independent features; comparing the higher-order pattern to ratings data associated to outcomes following one or more surgical procedures; and automatically generating a quality score based upon the comparing, wherein the quality score is predictive of an assessment of the technical skill and/or non-technical practice.

### CROSS REFERENCE TO RELATED APPLICATION

This application claims priority to and benefit of U.S. Provisional Application No. 62/802,293, filed on Feb. 7, 2019, entitled “Automated Identification and Grading of Intraoperative Quality,” the entire contents of which is hereby expressly incorporated herein by reference.

### STATEMENT OF GOVERNMENT SUPPORT

This invention was made with government support under HL146619 awarded by the National Institutes of Health. The government has certain rights in the invention.

### TECHNICAL FIELD

The present disclosure is generally directed to automated identification and grading of intraoperative quality, and more specifically, to automated identification and assessment of technical skills and/or non-technical practices exhibited by medical and/or other health professionals during a surgical operation using modeling and/or machine learning techniques.

### BACKGROUND

Complications arise from surgery, unfortunately. Conventionally, to evaluate surgical operations to determine the cause of complications, peer surgeons typically rate the “technical skills” of the surgeon(s) during the surgery under evaluation. There are several pitfalls however with the conventional approach.

First, other factors besides sheer technical skills may affect a patient's risk of developing a complication that arises from surgery. For instance, and particularly for surgical operations that require not only a surgeon but several other team members to work with the surgeon, non-technical practices may affect surgical outcomes. The performance of even an experienced surgeon, who may be fixed on the field of activity at hand, may be affected by background noise in the operating room or lack of closed loop communication with other team members. The number of distractions or breakdowns in communication in the operating room may affect complication outcomes, and generally, consistent patterns of distractions/breakdowns there are happening in the operating room may be a likely predictor of complications that may arise. To date, acquiring, analyzing, and incorporating the assessment of such “non-technical practices” into the evaluation of surgical operations have largely remained under-utilized.

Second, evaluation of a surgeon's technical skills are typically performed by peer surgeons who may exhibit bias, as peer surgeons tend to believe that their way of performing surgery is the correct way. Evaluations from peer reviewers may also be biased by factors unrelated to the surgeon's technical skills or ability to manage non-technical practices.

Third, there has been limited application of employing computer-assisted platforms to address the aforementioned limitations and in automating time-intensive human activities within the healthcare sector to address threats of objectivity and scalability within existing assessment approaches. Conventional computer-assisted platforms to date have focused on evaluating technical skills in simulated environments that fail to mimic live patient situations and non-technical practices that occur in an operating room. Such computer-assisted platforms are not configured to process data from a video and/or audio recording of an operation in a real environment in a way that is meaningful to characterize or otherwise recognize that the video and/or audio data is related to the technical skills and/or non-technical practices that may have contributed to a patient's development of a complication.

### BRIEF SUMMARY

Generally, a computing device may be configured to analyze a video and/or audio recording of a medical operation captured from a real (i.e., not simulated) environment, and further, utilize repeatable, automated, quantitative methods to generate model(s) to accurately characterize or otherwise recognize that the video and/or audio data is related to or is otherwise indicative of technical skills and/or non-technical practices of medical or health professionals. In some instances, the computing device may employ machine learning techniques, including but not limited to support vector machines (SVMs), ensemble classifiers, and artificial neural networks (ANNs), k-nearest neighbor, gradient boosting machine, Naive Bayes classifiers, linear convex kernels, random forest, and/or other suitable machine learning techniques, to learn how to model technical skills and/or non-technical practices and subsequently assess the underlying technical skills and/or non-technical practices from the models.

In one aspect, a computer-implemented method for characterizing and evaluating surgical procedures may include: (i) receiving one or more segments of a digital recording, wherein the one or segments include video and/or audio data of the surgical procedure; (ii) analyzing, via a video/audio understanding model, the one or more segments to (a) characterize a plurality of independent features associated with a technical skill and/or non-technical practice that are evident in the one or more segments and (b) determine a higher-order pattern based upon analyzing a group of at least two of the plurality of independent features; (iii) comparing the higher-order pattern to ratings data associated to outcomes following one or more surgical procedures; and (iv) automatically generating a quality score based upon the comparing, wherein the quality score is predictive of an assessment of the technical skill and/or non-technical practice.

In another aspect, a device for characterizing and evaluating surgical procedures may include: one or more processors; and an application comprising a set of computer-executable instructions stored on one or more memories, wherein the set of computer-executable instructions, when executed by the one or more processors, cause the one or more processors to: (i) receive one or more segments of a digital recording, wherein the one or segments include video and/or audio data of a surgical procedure; analyze, via a video/audio understanding model, the one or more segments to (a) characterize a plurality of independent features associated with a technical skill and/or a non-technical practice that are evident in the one or more segments and (b) determine a higher-order pattern based upon analyzing a group of at least two of the plurality of independent features; compare the higher-order pattern to ratings data associated to outcomes following one or more surgical procedures; and automatically generate a quality score based upon the comparing, wherein the quality score is predictive of an assessment of the technical skill and/or non-technical practice.

### BRIEF DESCRIPTION OF THE FIGURES

The figures described below depict various aspects of the system, apparatus, and methods disclosed therein. It should be understood that each figure depicts one embodiment of a particular aspect of the disclosed system, apparatus, and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 depicts an exemplary computing environment in which identification and/or assessment of technical skills and/or non-technical practices is performed, according to one embodiment;

FIG. 2 depicts an exemplary server by which technical skills and/or non-technical practices are recognized and/or evaluated, according to one embodiment;

FIG. 3 depicts an exemplary configuration of digital recording devices in an operating room, according to one embodiment;

FIGS. 4A-4B depict exemplary image frames associated with technical skills and/or non-technical practices that are identified and/or evaluated, according to one embodiment;

FIG. 5 depicts a flow diagram by which technical skills and/or non-technical practices are recognized and/or evaluated, according to one embodiment;

FIG. 6 depicts exemplary tables of various data associated with technical skills and/or non-technical practices that are recognized and/or evaluated, according to one embodiment; and

FIG. 7 depicts a flow diagram by which a digital recording is divided into a plurality of segments, according to one embodiment.

The figures depict preferred embodiments for purposes of illustration only. One skilled in the art will readily recognize from the following discussion that alternative embodiments of the system, apparatus, and methods illustrated herein may be employed without departing from the principles of the invention described herein.

### DETAILED DESCRIPTION

Generally, embodiments of the present invention solve the challenges identified above in the Background by analyzing, via a computing device executing a video/audio understanding model, real (i.e., not simulated) surgical operations recorded by a digital recording device (e.g., a video camera having a microphone) to assess technical skills and/or non-technical practices that occurred in the surgical operations. The technical skills and/or non-technical practices may be associated with postoperative complications. The assessments may be used for quality improvement initiatives, educating surgeons and other medical or health professionals, such as perfusionists, nurses, physician assistants, technicians, and credentialing of clinical providers for instance. Further, the assessments of technical skills and/or non-technical practices may be made in real-time if the digital recording of the operation is received in real-time (i.e., as the operation is occurring in real-time), thereby enabling the computing device to predict errors or prevent complications that may otherwise occur without predictive analytics capabilities.

In some embodiments, establishing the video/audio understanding model involves providing a machine-learning algorithm with training data to learn from during the training process. Generally, training data may contain labels of the correct answer (i.e., target attributes). The learning algorithm finds patterns in the training data that map the input data attributes to the target attributes, to output the machine-learning model that captures these patterns. Accordingly, the computing device can use the machine-learning model to generate predictions on new data for which the target attributes are unknown.

Specifically, in order to characterize the technical skills and/or non-technical practices, the data processed by the computing device as described herein captures a holistic view of a surgical operation. Therefore, the data may include a digital recording (e.g., acquired by a video and/or audio recording device) of a real (i.e., not simulated) surgical environment that captures technical skills and/or non-technical practices that have occurred in the surgical environment (i.e., operating room). The computing device is enabled to, via the video/audio understanding model, characterize independent features related to technical skills and/or non-technical practices that contribute to a complication, and further determine a higher-order pattern based upon analyzing a group of independent features. For example, upon analyzing video data from one or more video segments, the computing device may characterize independent features (e.g., pertaining to economy of motion of a surgeon's hand) related to a technical skill (e.g., suturing), such as a total distance travelled by the hand or tool held in the hand, frequency of suture knots tied (e.g., one knot), an amount of time taken for a suturing procedure, by analyzing image frames based on spatial dependencies and regional intensity levels of image pixels. Subsequently, the computing device may determine a higher-order pattern, such as suturing efficiency, by measuring the total distance travelled by the hand/tool with respect to the amount of time taken for a suturing procedure, or mean velocity of a scalpel. Fewer unnecessary movements of the hand (i.e., shorter distance travelled by the hand) during the span of time to perform the suture may be a higher-order pattern corresponding to more efficient suturing, for example. As another example, upon analyzing audio data from one or more video segments, the computing device may characterize independent features (e.g., pertaining to intensity of verbal cues) related to a non-technical practice (e.g., communication with a team member), such as a frequency and/or volume of a particular word or phrase, and duration of a surgical procedure. Verbal cues may be associated with natural language (i.e., actual words spoken) or sentiment delivery (i.e., how words were spoken, such as the volume of the voice). Subsequently, the computing device may determine a higher-order pattern, such as team conflict percentage, by measuring the total number of verbal cues indicating conflict (e.g., as indicated by loud voices) with respect to the duration of a phase of a surgical procedure, mean volume or mean frequency (represented by a spectrogram, Mel Filterbank (MFB)) corresponding to verbal cues by a physician assistant.

In some embodiments, computational techniques leveraging classical modeling (e.g., Lucas-Kanade technique) for tracking movement (e.g., of a surgeon's hand or tool, of a nurse's head nodding as a signal of communicating affirmation to a surgeon) may be used to develop the video/audio understanding model that characterizes a plurality of independent features associated with a technical skill and/or a non-technical practice that are evident in the one or more segments of a digital recording.

In some embodiments, machine-learning techniques may be used to generate the video/audio understanding model. For example, a computing device with unsupervised machine learning capabilities may train the video/audio understanding model by analyzing raw segments of a digital recording (i.e., no labels) to characterize a plurality of independent features associated with a technical skill and/or a non-technical practice.

As another example, using annotation software installed at a data analysis platform equipped with video/audio playback software and/or data visualization software human reviewers may view video and/or audio data of a surgical procedure captured in segment(s) of a digital recording, label the features evident within the segments, and/or rate the segment(s) with a peer rating score based on standard grading criteria as known in the medical field. It should be noted that digital recording segments provided to human reviewers for labeling may represent critical actions during particular phases (e.g., pre-incision timeout, incision, suturing) of the operation, and are preferably short in duration (e.g., less than one hour), so that particular segments, as opposed to the entire digital recording, can be efficiently and timely peer-reviewed (e.g., by surgeon, anesthesiologist, or perfusionist). In some instances, the reviewers may refer to Electronic Health Records (EHR) data corresponding to the one or more segments to facilitate their review. The labeled features (i.e., training data) may be provided to the computing device with supervised machine learning capabilities to enable the computing device to train the video/audio understanding model to characterize the features labeled by the human reviewers and subsequently determine a higher-order pattern upon analyzing a group of at least two of the independent features. In some embodiments, the digital recording may be time-synchronized with EHR data, in order to create a richer dataset used to associate behaviors/actions observable in recordings with factors observable in the EHR (e.g. hemodynamic derangements detected from physiologic monitors, medications administered). Such a dataset may assist in training the video/audio understanding model to characterize intraoperative events (i.e., technical skills and/or non-technical practices). The computing device may also automatically evaluate the higher-order pattern. For example, the computing device may generate a quality score by comparing the higher-order pattern to ratings data (e.g., the peer rating score established by a human reviewer mentioned above). Further, to predict whether a complication may result from the higher-order pattern, the computing device may further associate the higher-order pattern with outcomes (e.g., complications) following surgery. Such complications information may be retrieved from participating hospitals or a proprietary database configured to store patient outcome data, such as the Society of Thoracic Surgeons Adult Cardiac Surgery Database. Complications information may also be evident in EHR data. EHR data may also contain minute-to-minute statuses that provides context of what is actually going on during the surgical operation.

The digital recording may have been produced using any standard known in the art, such as HDTV high-definition video modes like 1080p, and the duration of some surgeries may last several hours. Feeding the entire unstructured digital recording without EHR data and any additional training data for training a machine-learning computing device to characterize technical skills and/or non-technical practices contained throughout the entire digital recording may be computationally burdensome. Therefore, in some embodiments, the training data fed into the machine-learning computing device for training purposes may not only comprise the digital recording, but also additional file(s) that include annotations (e.g., time stamps, frame numbers) that indicate which portions of the digital recording include clinically relevant segments, so that the machine-learning computing device system may automatically splice the digital recording into clinically relevant segments using the annotations, and subsequently characterize features from the more manageable digital recording segments in a meaningful and structured way.

In some embodiments, the training data fed into the machine-learning computing device for training purposes may not only comprise the digital recording, but also additional file(s) that include EHR data temporally synchronized (i.e., time stamped) with the digital recording, so that the machine-learning computing device may automatically splice the digital recording into clinically relevant segments using the EHR data, and subsequently characterize features from the digital recording segments in a meaningful and structured way.

Therefore, the machine-learning computing device need not expend computational resources on processing the entire digital recording, and instead, may focus its resources on processing portions (segments) of the digital recording that portray clinically relevant activity. Accordingly, scalability of the machine-learning computing device is possible.

The machine-learning computing device described above may, inter alia, analyze and characterize recording data, using an architecture composed of various types of machine learning models, such as ensemble classifiers, ANNs (e.g., convolutional neural networks (CNNs), recurrent neural networks (RNNs), etc.), where the machine learning models may analyze the recording data to determine or predict a set of surgical phases that may be depicted or otherwise included in the recording data. The machine learning models may be configured to characterize independent features, determine patterns or correlations between complex, nonlinear and hidden relationships among the independent features representing technical skills and/or non-technical practices, and rate the patterns of surgical phases.

Specifically, machine learning may be used to train a computer to recognize patterns inherent in evaluated technical skills and/or non-technical practices. Those patterns may be used to analyze and characterize recording data portraying the technical skills and/or non-technical practices. Machine learning (ML) models may be trained with training data relevant to surgical operations, using back-propagation or other training techniques. In particular, recording data may be input into models, which may analyze the inputted data to arrive at a prediction. By recursively arriving at predictions, comparing the predictions to the training labels, and minimizing the error between the predictions and the training labels, the corresponding model may train itself. According to embodiments, the trained model may be configured with a set of parameters which enable the trained model to analyze unseen recording data.

Exemplary Computing Environment

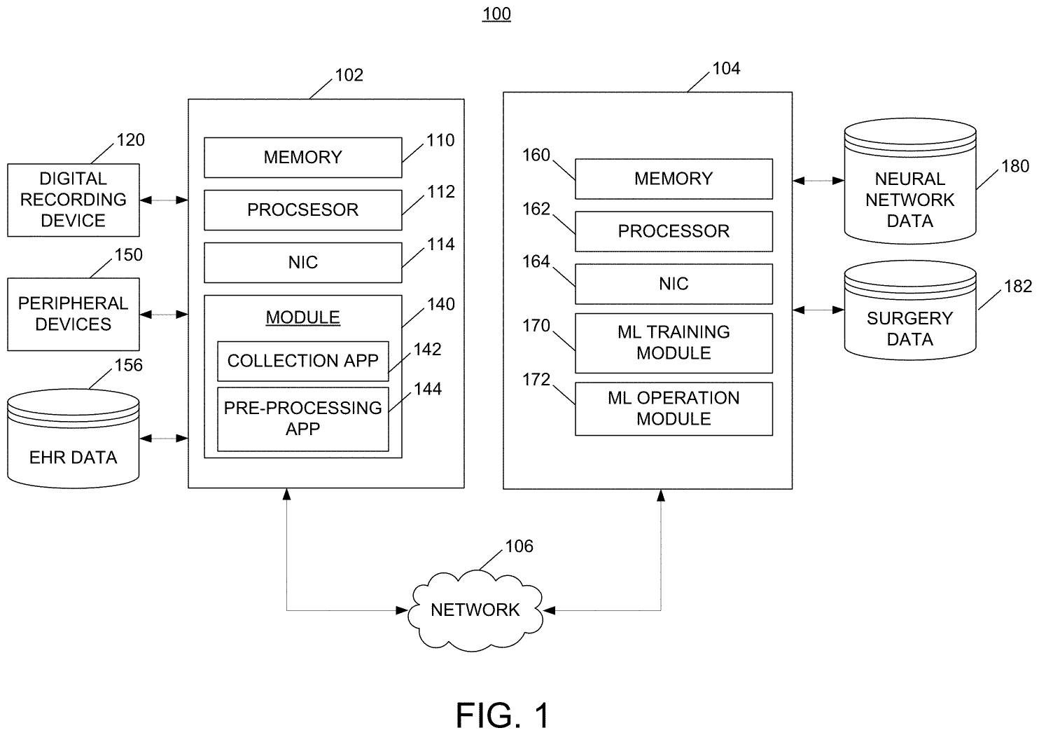

FIG. 1 depicts an exemplary computing environment 100 configured to perform identification and/or assessment of technical skills and/or non-technical practices. The computing environment 100 may generally include any combination of hardware, software, and storage elements, and may be configured to facilitate the embodiments discussed herein. Particularly, environment 100 may include a computing system comprising a client 102 and a server 104, each of which may be communicatively coupled by a network 106. Client 102 and/or server 104 may, respectively, be any suitable computing device such as a server device, laptop, smart phone, tablet, wearable device, etc. Network 106 may comprise any suitable network or networks, including a local area network (LAN), wide area network (WAN), Internet, or combination thereof.

Client 102 may include a memory 110 and a processor 112 for storing and executing, respectively, a module 140. Memory 110 may include one or more suitable storage media such as a magnetic storage device, a solid-state drive, random access memory (RAM), etc. Processor 112 may include one or more suitable processors (e.g., central processing units (CPUs) and/or graphics processing units (GPUs)). Client 102 may also include a network interface controller (NIC) 114. NIC 114 may include any suitable network interface controller(s), to enable client 102 to communicate over network 106 via any suitable wired and/or wireless connection. Digital recording device 120 may be a purpose-built or commercially available digital recording device, and may be integral to client 102 or external to client 102. Digital recording device 120 may be coupled, communicatively and/or physically, to client 102, and may include mechanisms for recording a surgical operation (e.g., an image sensor, a microphone) and outputting the digital recording (i.e., recording data) to processor 112, for example.

Recording data may be various types of real-time or stored media data, including digital video data (which may be composed of a sequence of image frames), image data, audio data, or other suitable data. In one implementation, the client device 102 or digital recording device 120 may transmit the digital recording data to the server 104 in real-time or near-real-time as the digital recording data are generated. In another implementation, the client device 102 or digital recording device 120 may transmit the digital recording data to the server 104 at a time subsequent to generating the digital recording data, such as in response to a request from the server 104. The server 104 may store the recording data locally or may cause the surgery database 182 to store the digital recording data.

Module 140, stored in memory 110 as a set of computer-readable instructions, may include a collection application 142 and/or pre-processing application 144 which when executed by processor 112 cause recording data and/or metadata to be retrieved or read from digital recording device 120, modified, and/or stored in memory 110. Client 102 may include peripheral devices 150 by which a user may, respectively, enter input and receive output. In some embodiments, peripheral devices 150 may be integrated, such as in a touch screen device. Client 102 may also be communicatively coupled to an EHR database 156. As will be further described below, the pre-processing application 144 may modify the recording data by temporally synchronizing it with EHR data received from the EHR database 156.

Server 104 may include a memory 160 and a processor 162 for storing and executing, respectively, modules. Server 104 may also include a NIC 164, which may include any suitable network interface controller(s), to enable server 104 to communicate over network 106 via any suitable wired and/or wireless connection. In some embodiments, modules may include a machine learning (ML) training module 170 and a ML operation module 172. Each of the modules 170, 172 may be stored, respectively, in memory 160 as a set of computer-readable instructions. When executed by processor 162, the set of instructions corresponding to ML training module 170 may generate or otherwise receive training data to train models, so that models may cause surgical procedures to be characterized and assessed. When executed by processor 162, the set of instructions corresponding to ML operation module 172 may cause recording data to be input to a trained model, may cause the model to be operated, and may cause data to be stored to memory 160 or another location.

In embodiments, ML training module 170 may train one or more neural networks to receive and process recording data, such as recording data produced by digital recording device 120. First, ML training module 170 may generate a training data set with many (e.g., tens of thousands or more) labeled surgical phases that are plausible to occur in any given surgical procedure. The labeled surgical phases may be based upon real operations that have been recorded.

Particularly, training data may include recording segments each corresponding to a particular surgical phase of a surgical procedure, along with an appropriate label (e.g., the type of procedure, an evaluation of the technical skills and/or non-technical practices within the procedure). For example, a surgical procedure related to heart surgery may include at least a surgical phase related to arterial cannulation and another surgical phase related to communication between team members regarding the onset of cardiopulmonary bypass (for cardiac surgical procedures involving cardiopulmonary bypass).

ML training module 170 may create a tiered, and/or hierarchical, model wherein the root element of the model comprises a classification model (e.g., a multi-layer perceptron feed-forward neural network) trained using the training data set as training input to classify recording data according to the type of procedure and an evaluation of the procedure. In an embodiment, the model, or parts thereof, may be constructed using a compiled programming language for faster execution. The model may be trained using supervised learning. Branching from the root element may be regression models that ML training module 170 may train to predict parameters based on recording data. ML training module 170 may train regression models individually for each distinct type of surgical phase and surgical procedure. Model data 180 may store the trained hierarchical model, comprising trained classification model and one or more trained regression models.

After ML training module 170 fully trains the hierarchical model, a user of client 102 may request an analysis of a sample recording data by, for example, interacting with peripheral devices 150 (e.g., input devices, display devices). Collection application 142 may receive and/or retrieve the sample recording data and pre-processing application 144 may pre-process the recording data (e.g., synchronizing EHR data to the recording data) based on EHR data retrieved from an EHR database 156. Pre-processing may include other suitable operations, such as numerical formatting (e.g., rounding), data validation, alignment, etc. The recording data may then be persisted for later analysis by, for example, module 140 writing the data out to memory 110. Alternately, or in addition, the recording data may be transferred to another computer (e.g., server 104) for further analysis (e.g., by a trained model) via network 106. Although the foregoing operation includes a user, in some embodiments, recording data analysis may be requested/initiated via automated (e.g., robotic) means.

In some embodiments wherein the recording data are transmitted to, and/or retrieved by server 104, the recording data may be immediately input into a trained model. For example, in an embodiment, ML operation module 172 may include instructions that, when executed by processor 162, cause a trained model to be retrieved from model data 180. The instructions may further include retrieving the recording data produced by digital recording device 120, and passing the recording data to the trained model. The data may be passed all at once or in chunks (e.g., in real-time as the data are produced). The trained model may then process the input provided by ML operation module 172 to divide the recording data into segments.

Once the trained model has divided the recording data into recording segments, the trained model may characterize and evaluate the surgical phase corresponding to the recording segment. ML operation module 172 may include computer-readable instructions that, when executed by processor 162, selects the results of the evaluation and transmits the evaluation (e.g., a quality score) back to the user, and/or stores the results in association with the recording data.

Although FIG. 1 depicts a client 102 and a server 104 in communication via an electronic computer network 106, in some embodiments, the client 102 and the server 104 may be combined into a single device. In some embodiments, ML operation module 172 may be located in client 102. The client/server architecture, or lack thereof, may depend on the needs of particular applications. For example, in some applications of the technology described herein, network latencies may be unacceptable. As another example, ML training module 170 may train a model in server 104, and serialize and/or store the trained model in memory 160 and/or model data 180. The trained model may then be transmitted by server 104 to client 102, and/or retrieved by client 102. Once retrieved by client 102, an ML operation module 172 located in client 102 may operate the trained model.

Exemplary Server

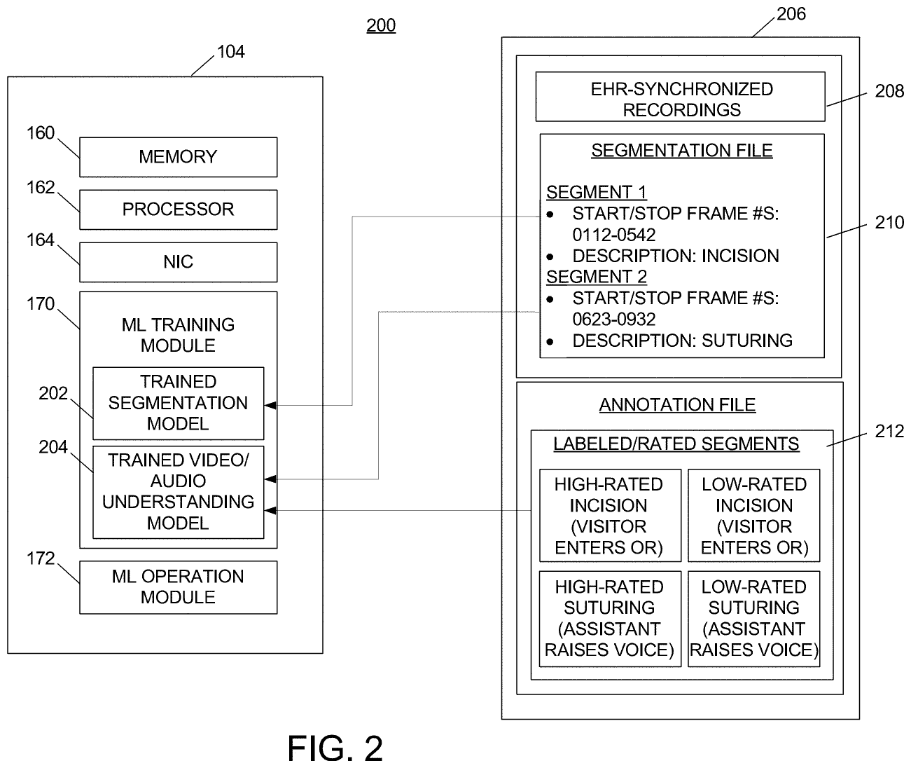

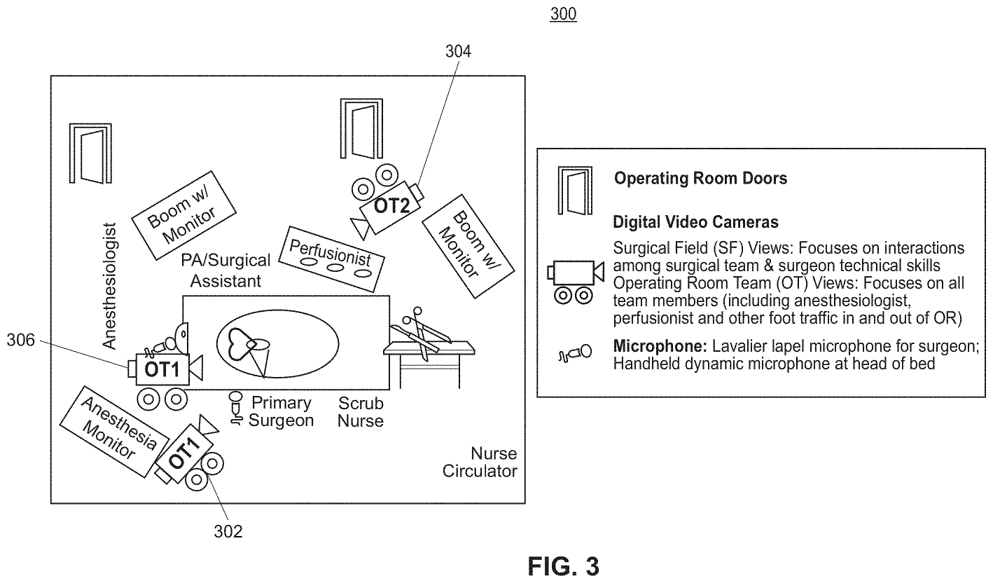

Turning now to FIG. 2, an exemplary server 104 is shown. The server 104 (e.g., via processor 162) may receive a digital recording of a particular operation. The digital recording may be produced by one or more digital recording devices 120 (e.g., a video camera with a built-in microphone) placed in an operating room 300, as shown in FIG. 3. For example, digital recording device 302 may record interactions between a surgeon, physician assistant (PA), and/or a nurse. Digital recording device 304 may record interactions between a perfusion team member and another surgical team member. Digital recording device 306 may record the entire surgery team and operation room, which may capture any foot traffic in and out of the operating room, for example. One of ordinary skill in the art will recognize that additional or less digital recording devices may be used, and that the digital recording devices may be positioned in various areas with appropriate levels of zoom to capture various scenes of the operating room. If multiple digital recording devices are used, the respective digital recordings produced may be merged into one digital recording for analysis by the server 104. Alternatively, server 104 may analyze the respective digital recordings produced individually in a coordinated manner. For ease of illustration and explanation, a single digital recording and EHR data file will be referred to throughout the disclosure with respect to server 104 as a non-limiting example.

A computing device (e.g., client device 102 of FIG. 1) equipped with pre-processing software (e.g., pre-processing app 144 of FIG. 1) may receive a digital recording from the digital recording device and an EHR data file that corresponds to the operation recorded in the digital recording from an EHR system. The EHR system may convert or otherwise receive precise documentation data collected by observers (e.g., circulating nurses, monitoring surgeons, etc.) of the operation. Documentation data may include information gathered on the type of procedure, intraoperative documentation times, procedure start and stop times, number of staff in the operating room, minute-to-minute statuses that provides context of what is actually going on during the surgical operation, any complication(s) that may have been caused by the operation, or any suitable information representative of the operation. The computing device, via the pre-processing software, may synchronize the digital recording with the EHR data file temporally to produce an EHR data-synchronized digital recording. As such, the digital recording may be time-synchronized with EHR data. The digital recording that is time-synchronized with EHR data may provide a rich dataset of features used to improve prediction of downstream complications by the server 104. For example, features related to technical skills (e.g., slower speed of the surgeon in operating) led to prolonged exposure to cardiopulmonary bypass, leading to a greater degree of bypass-induced inflammation to the kidneys, which led to, at least in part, a complication (e.g., acute kidney injury manifesting 24 hours after the surgery). As another example, features related to non-technical practices (e.g., poor communication between the surgeon, anesthesiologist, and perfusionist when transitioning on and off cardiopulmonary bypass) led to episodes of low blood pressure (hypotensive episode), which led to, at least in part, a complication (e.g., acute kidney injury manifesting 24 hours after the surgery).

Upon receiving the EHR data-synchronized digital recording, the server 104, via a trained segmentation model 202, uses the EHR data to automatically splice or parse the EHR data-synchronized digital recording to extract meaningful recording segments that capture clinically relevant aspects of an operation. That is, the server 104 may be capable of segmenting a long, unconstrained digital recording into segments using the EHR data that has been synchronized with the digital recording. For example, meaningful recording segments may show when an operation team is discussing a plan for a certain step of the operation, a scalpel is applied to a patient to begin an incision, a patient is actually being connected to a heart/lung machine, etc. Examples of recording segments that may not be clinically relevant to evaluate technical skills and/or non-technical practices may be at the beginning and end of an operation, such as preparing the patient for surgery or recovery time. The segmentation model 202 may generally be implemented or trained to identify clinically relevant aspects of an operation via computational or machine learning techniques applied to recording segments labeled as clinically relevant, including but not limited to SVMs, ensemble classifiers, and ANNs, such as a RNN or a Long Short-Term Memory (LSTM) network.

Specifically, the server 104, via the segmentation model 202, may be configured to encode frames of the EHR data-synchronized digital recording into embedding vectors. As the frames have been temporally synchronized with EHR data, each vector corresponding to a frame may include at least one designated EHR value that represents EHR data that describes the frame, such as a nurse's gaze direction (e.g., designated with a value of “1”), a description of a motion of a surgical tool (e.g., designated with a value of “2”), a location description of a surgeon's hands (e.g., designated with a value of “3”), for example. Other representations are contemplated. Each vector may also include values representative of spatial dependencies and regional intensity levels of image pixels. By encoding frames into embedding vectors and processing the embedding vectors in subsequent stages instead of the frames themselves, the server 104 may process a manageable amount of data. That is, analyzing the full EHR data-synchronized digital recording without encoding frames into vectors generally would require a large amount of memory and computation power. It should be recognized that although vector representation is illustrated, such example should not be considered limiting. Other suitable data representations are contemplated, such as a tensor representation.

The server 104, via the segmentation model 202, may also be configured to analyze a sequence of embedding vectors to propose plausible recording segments based on the values contained in the embedding vectors. That is, the segmentation model 202 utilized by the server 104 may learn to classify image frames as clinically relevant based on the associations of the EHR value and other values contained in each vector.

The server 104, via the segmentation model 202, may also be configured to select, among the proposed segments, a group of recording segments that are likely to exhibit a sequence of technical skills and/or non-technical practices representative of a surgical phase, based on temporal dependencies among the proposed segments. Accordingly, the segmentation model 202 may learn how certain surgical phases of a surgical procedure that involve technical skills and/or non-technical practices are staged in sequence.

To assess the accuracy of the segmentation model's proposal capabilities, the segmentation model 202 may receive a segmentation file that includes recording segments identified by their start and stop frame numbers and further labeled with a suitable description (e.g., “suturing,” “cannulating aorta,” “repeating an instruction for verification,” “turning on ventilator,” “turning on cardiopulmonary bypass pump,” “making an incision,” etc.) indicating which segments show clinically relevant activities during training, for example. Further improvements to the segmentation model 202 may be made based on a comparison of the proposed segments and labeled segments.

At the beginning of training, the segmentation model 202 may be initialized with a random set of parameters, and the segmentation model 202 may iteratively refine them based on the (i) empirical performance (e.g., ability to propose and localize recording segments in an unseen EHR data-synchronized digital recording based on their visual appearance and temporal relations), and (ii) labeled recording segments. The segmentation model 202 may continuously learn so that segmentation of EHR data-synchronized digital recordings are as close to labeled recording segments as possible.

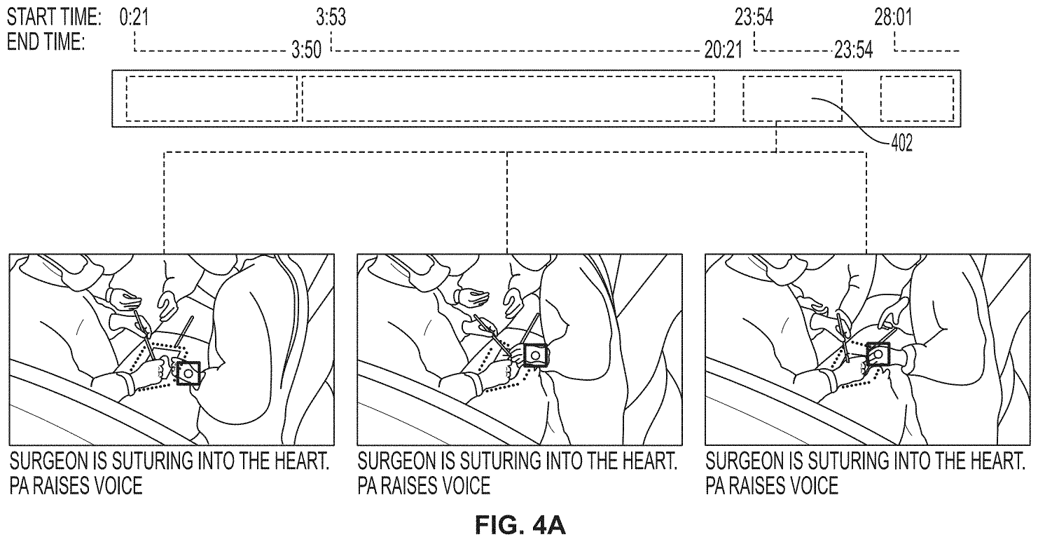

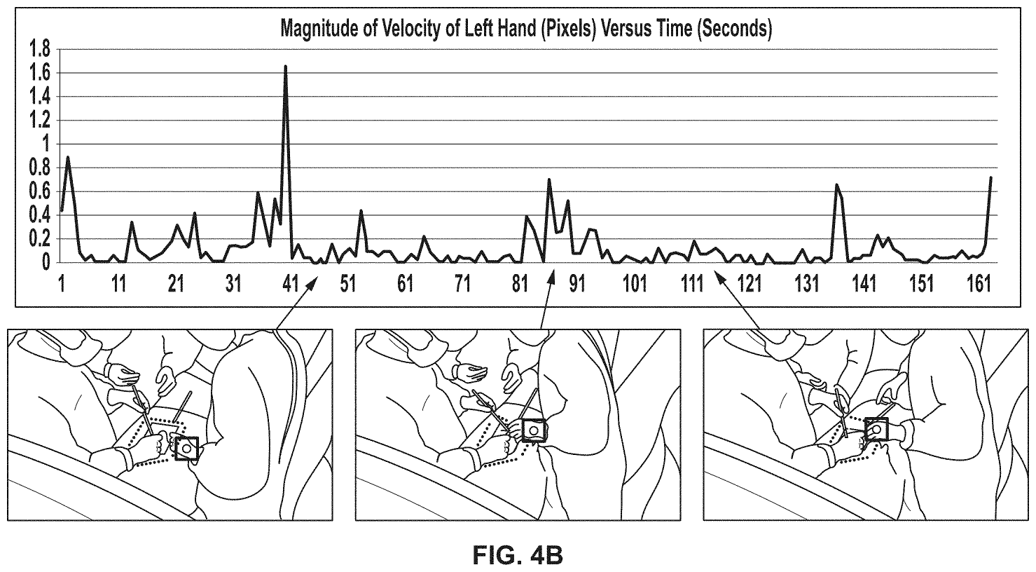

Training data may include a study dataset (i.e., a plurality of different EHR-synchronized digital recordings 208) and segmentation file(s) 210 that include supplemental information that identifies portions (i.e., recording segments) of the EHR-synchronized digital recordings that are meaningful. For example, the study dataset may be assigned to an annotation computing platform (e.g., client device 102) with segments capturing various phases of the operation accessible by peer raters, who may provide labels to features within the segments using annotation software installed at the annotation computing platform. The assignment of the study dataset may be handled in accordance with the annotation computing platform. In embodiments, each EHR data-synchronized digital recording may be assigned to a peer rater to annotate each EHR data-synchronized digital recording with temporal segment boundary annotations (e.g., start and stop frame numbers), and segmentation file(s) 210 may store such temporal segment boundary annotations. In some embodiments, a peer rater may also provide rich semantic information annotation in labels (e.g., a phrase or sentence describing the recording segments), which may provide richer context in addition to the EHR data that has been synchronized with the digital recording. The peer rater may also have access to audio data when scribing the labels. The labels may also be documented in the segmentation file(s) 210. As such, the recording segments may be temporally localized (e.g., with timestamps indicative of start and end temporal boundaries for each segment in a given digital recording) and/or described by labels, as shown in an example in FIG. 4A. Because labels may be provided at the segment level and not the frame level, the annotations may contain richer semantic information and better capture the surgical phases. Accordingly, in contrast to conventional models that model temporal dependencies at the frame-level, the segmentation model 202 aims to model temporal dependencies at the segment-level.

For the segmentation model 202 to propose segments based on L embedding vectors, first a set of candidate anchors and durations may be designed. These anchors and durations specify all possible segments and may be defined by hand or optimized via computational or learning methods. Second, a computational or learning-based method may be designed to apply the candidate anchors and durations to the L embedding vectors and iteratively extract the most plausible segments. Extraction and plausibility may be part of a computational process such a greedy optimization, a classical machine learning process such as Hidden Markov Models, or a deep-learning-based process such as Long-Short-Term-Memory (LSTM) networks.

The server 104, via a trained video/audio understanding model 204, may process the recording segments to automatically recognize and objectively evaluate technical skills and/or non-technical practices that are evident in the recording segments.

The trained video/audio understanding model 204 may be configured to determine high-dimensional (i.e., higher-order) patterns of the recording segments, upon characterizing groups of features and conducting audio-behavioral analysis (i.e., audio understanding). The trained video/audio understanding model 204 may extract groups of features associated with technical skills and classify groups of the features into various higher-order patterns, such as efficiency patterns of movement of a surgeon's hand or tool. Similarly, the trained video/audio understanding model 204 may extract groups of features associated with non-technical practices from the recording segments, and classify groups of features as various higher-order patterns, such as confrontational behavior based on a plurality of independent features (e.g., irritated facial expression, raising one's voice, etc.), team-supporting behavior based on a plurality of independent features (e.g., nodding, telling a health professional what to do with the patient, etc.), or any other suitable pattern related to an ethogram to quantify operating room behavior.

Video understanding generally focuses on characterizing and tracking objects over time from recording segments to understand the meaning inherent within pixels associated with moving images. This disclosure contemplates various video features or measures of surgical phases that a machine may actually be able to analyze. For example, to evaluate technical skills, video features may include various mean velocities of a surgeon's hand or surgical instrument across different phases of an operation. For instance, different mean velocities in the surgeon's hand may be identified when suturing a new valve into the patient's heart. To evaluate non-technical practices, video features may include the percentage of time that the anesthesiologist and/or surgeon focus on the anesthesia hemodynamic monitors during critical portions of an operation, the number of times the operating room doors open per hour over phases of an operation, or other team behaviors not directly related to surgical technique or use of medication, etc.

Audio understanding generally focuses on characterizing audio that is included in recording segments to understand the meaning inherent within the audio that corresponds to the moving images. The identified audio may be analyzed alone, or in relation to the corresponding moving images. Audio analysis is particularly important for evaluation of non-technical practices, since communication between team members is a critical domain of non-technical practices. Audio may be depicted by spectrogram(s), which represents a visual spectrum of frequencies included in a sound. Spectrogram(s) may include multiple dimensions corresponding to time, frequency, and amplitude of a particular frequency. It has been found that speech may be analyzed to recognize mood patterns and to measure a subject's behavior. A subject's mood state may be predicted by using (i) acoustic features common to emotion classification tasks, (ii) features that capture speech rhythm, and (iii) creating person-dependent representations via personal call data, such as via captured audio during phone conversations during daily routines. For example, verbal communication (e.g., how and what was said, interpersonal dynamics, timings and delays between responses, cognitive load) may be analyzed to assess non-technical practices. The manner in which a surgeon communicates may also affect how others perceive his/her abilities as captured through paralinguistic properties of spoken behavior (e.g., emotion, fatigue, stress, frustration, etc.).

Accordingly, this disclosure contemplates various audio features or measures of surgical phases that a machine may actually be able to analyze. For example, to evaluate technical skills and/or non-technical practices, audio features may include spectrogram(s) associated to (i) lingual types of audio signals (e.g., using words to communicate information), which happens in operating room and (ii) non-lingual types of audio signals (e.g., tenor of someone's voice, volume, gaps in interaction, etc.) as potential factors that could impact non-technical practices. It should be recognized that in some embodiments, analyzing audio may not be required when characterizing or evaluating technical skills and/or non-technical practices, particularly when analyzing non-verbal communication (e.g., transferring of instruments between team members as a proxy for decision making) to assess non-technical practices, or when analyzing surgeon movements (e.g., instrument handling) to assess technical skills.

At the beginning of training, the video/audio understanding model 204 may be initialized with a random set of parameters, and the video/audio understanding model 204 may iteratively refine them based on the empirical performance (e.g., ability to detect and rate features in unseen recording segments), and (ii) labeled segments. The video/audio understanding model 204 may continuously learn so that extraction of video and audio features (and ratings thereof) are as close to labeled segments as possible.

To train the video/audio understanding model 204, training data, such as labeled (with ratings) segments included in an annotation file 212, may be generated by the peer rating platform (e.g., client device 102) mentioned above. A plurality of unlabeled and unrated recording segments may be accessible by peer raters that may provide technical and non-technical assessments of the recording segments based on the video and audio observed. The peer rating platform may be configured to facilitate objective feedback from the peer raters. For example, each recording segment may be assigned to a fixed number of raters, each rating technical skills and/or non-technical practices. The peer rating platform may calculate a score (e.g., mean, median, mode, range, delta, etc.) representative of the ratings provided by some or all of the raters for each recording segment. Such score may be associated with ratings data associated to outcomes following one or more surgical procedures, such as the Society of Thoracic Surgeons (STS)'s composite major complication rate (e.g., permanent stroke, surgical re-exploration, deep sternal wound infection, renal failure, prolonged ventilation or operative mortality). To standardize and objectify the peer rating process, peer raters may use a common validated assessment tool to rate each recording segment. For instance, raters may use Objective Structured Assessment of Technical Skills (OSATS) via a five-point behaviorally anchored scale, the domains of which may include respect for tissue, time and motion, instrument handling, and flow of operation to evaluate technical skills. To rate non-technical practices, raters may use Non-Technical Skills for Surgeons (NOTSS) via a validated four-point ordinal scale, the domains of which may include situation awareness, decision making, communication and teamwork, and leadership.

Other techniques to further objectify the ratings may include resubmitting a certain percentage (e.g., 20%) of the recording segments for review. A technique for minimizing intra-peer rater variability may include using linear mixed effect models to model ratings of operations where peer raters and surgeons are included as random effects. The fit of the linear mixed effect models may be used to quantify variation in the ratings by calculating an intra-class correlation coefficient to measure inter-peer rater reliability.

Qualified peer raters (e.g., surgeons) may assess many recording segments depicting surgical phases to rate a surgeon's technical skills and an operative team's non-technical practices. Peer raters may provide domain-specific and an overall summary assessment for each recording segment. In some embodiments, the peer raters may provide bounding-box labels for each feature identified in each recording segment. For example, in viewing a recording segment portraying suturing, a peer rater may provide bounding-box labels (including a rating) for a feature depicting the economy of motion (e.g., mean velocity of the suturing hand). As another example, a peer rater may provide bounding-box labels (including a rating) for aspects depicting communication and teamwork (e.g., average energy in each provider's sentences over the course of a particular surgical phase, such as initiation of bypass) or flow disruptions (e.g., number of door openings per hour over a particular surgical phase, number of personnel, other than team members, entering and leaving the operating room). By labeling the features, peer raters play an important role in converting features (e.g., motion information, sound information) contained in recording segments into data structures readable by a computer to characterize the features extracted from the recording segments.

Upon completion of peer rating the recording segments, each recording segment may be associated or otherwise labeled with an objective rating (i.e., the gold standard peer rating) of the surgeon and/or operative team based on ratings provided by the peer raters. The voluminous labeled recording segment may be collected and stored as training data.

Using the training data, the video/audio understanding model 204 may be developed using classical machine learning, such as boosting (e.g., for cases of limited data), and deep learning (e.g., for cases with ample data) approaches so that the video/audio understanding model 204 may learn visual detection and visual tracking. Ambiguity reduction techniques may be applied across time-synchronized recording segments (e.g., the three time-synchronized recording segments shown in FIG. 4B) to harmonize (i.e., rather than duplicate) aspects within and across video angles to develop the video/audio understanding model 204. The video/audio understanding model 204 may subsequently begin to learn visual detection and tracking for both technical skills and/or non-technical practices. For example, the learned video/audio understanding model 204 may characterize an operative team member's head focused on the hemodynamic monitor (i.e., a non-technical practice) based on detection in a single video frame, an operative team member's gaze focused on the surgeon's hand and then anticipating what tool the surgeon will use next by shifting his gaze at an instrument tray (i.e., a non-technical practice) based on tracking of the detected gaze throughout the video frames, instrument exchanges by a surgeon's hand (i.e., a technical skill) or even between scrub nurse-surgeon-scrub nurse (i.e., a technical skill) based on tracking of the detected instrument throughout the video frames. For instance, to measure economy of motion for a surgeon's hands, the video/audio understanding model 204 may learn to detect the surgeon's hands at frame t, track the surgeon's hands at all future frames t+k, and then compute a trajectory of the centroid of the detected bounding boxes. The video/audio understanding model 204 may use both classical physics-based tracking techniques (e.g., Lucas-Kanade tracking) and modern deep-learning based techniques, and may characterize a number of features, including economy of motion (e.g., mean acceleration, variance of local change in the trajectory against a linear or smoothed trajectory).

As mentioned above, audio (which may be visually depicted in a spectrogram) that is included in the recording segments may also be analyzed to train the video/audio understanding model 204 to understand the meaning inherent within the audio that corresponds to the moving images. Accordingly, analyzing the audio to develop the video/audio understanding model 204 may be a valuable complement. The video/audio understanding model 204 may learn how to extract at least two types of speech features for a particular speaking team member: low-level and high-level. Low-level features may represent the speaking styles of team members (e.g., relative loudness, speech clarity (articulation), and pitch contour, such as raising or lowering pitch). In contrast, high-level features may represent communication dynamics (e.g., pause variability (how response time changes over time amongst members of the team), overlapping speech (interruptions), entrainment (how speech patterns become more/less similar over time), and individual variability (how have individual speaking styles changed over the course of the surgery).

Using the training data that includes identification of the technical skills and/or non-technical practices as annotated/labeled by peer raters, the video/audio understanding model 204 may be trained to characterize independent features. For example, a feature of a technical skill may be mean velocity of a surgical tool or a suturing hand, or any suitable economy of motion. A feature of a non-technical skill may be frequency of repeating instructions for confirmation or volume (in decibels) of the instructions. Characterized features may be verified and/or compared against labeled features from peer raters for accuracy during training of the video/audio understanding model 204.

The video/audio understanding model 204 may then determine a higher-order pattern based upon analyzing a group of independent features for each of the technical skill or non-technical practice. Depending on the type of higher-order patterns, different computational and machine-learning techniques may be applied. For example, if the pattern is temporal in nature than a Markov or a Hidden Markov Model may be applied, or even a Recurrent Neural Network. One such case would be the higher-order pattern capturing the rate of movement of the stitching apparatus or the hand. Such patterns of video understanding may then be compared to peer rater assessments (e.g., as provided by human raters using NOTSS, ANTS, PINTS, OSATS scoring systems), which may be associated to outcomes following surgery (e.g., permanent stroke, surgical re-exploration, deep sternal wound infection, renal failure, prolonged ventilation or operative mortality, STS complications).

Based on the comparison, the server 104 may automatically generate one or more quality scores predictive of an assessment of the technical skills and/or non-technical practices in the recording segments. For example, server 104 may correlate the evaluated technical skills and/or non-technical practices to objective metrics, such as those provided by OSATS and NOTSS. The video/audio understanding model 204 may be improved by comparing the generated quality scores with the ratings provided by the peer raters.

As described herein, both the segmentation model 202 and video/audio understanding model 204 may be trained using supervision techniques with respect to training data 206. In other embodiments, the video/audio understanding model 204 may be trained using weak supervision techniques. For instance, a weakly supervised model may utilize EHR data and a limited range of segment labels (i.e., instead of receiving the full range of supervision as needed to train a supervised model) to characterize and rate technical skills and/or non-technical practices contained in the recording segments proposed by the segmentation model 202. Stated differently, the weakly supervised model may not only be learning that various ratings of identified technical skills and/or non-technical practices are contained in labeled segments, but may also be learning patterns in the characterized technical skills and/or non-technical practices that led to the various ratings, and applying the pattern recognition to the proposed recording segments to technical skills and/or non-technical practices that are contained in the proposed recording segments.

Although one server 104 is shown, additional servers may be used. For example, a server may be dedicated to recognizing and assessing technical skills, and another server may be dedicated to recognizing and assessing non-technical practices.

Exemplary Method

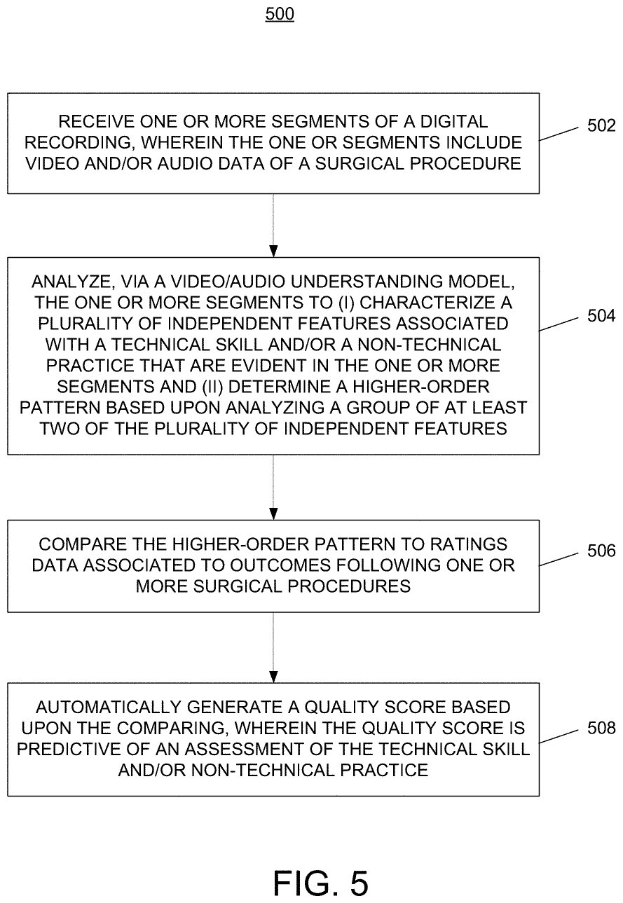

FIG. 5 depicts a flowchart describing a method 500 to automatically recognize and objectively assess technical skills and/or non-technical practices, in an embodiment. Method 500 may be performed by the server 104.

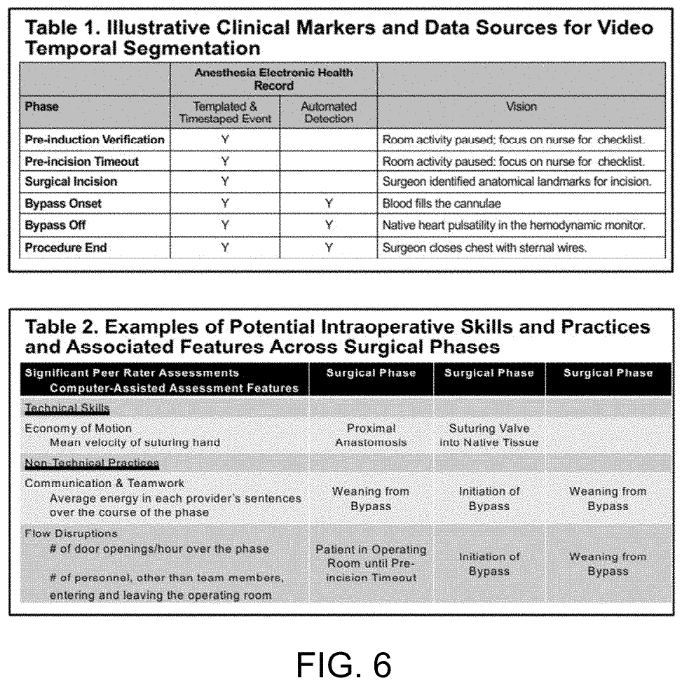

As shown, method 500 may begin by receiving one or more segments of a digital recording of a surgical procedure, as shown in block 502. The one or more segments may include video data and/or audio data depicting an actual surgical operation. Real-time capture of a digital recording may begin with the patient arriving at the operating room and end with the patient exiting the operating room. In one example, digital recording devices (e.g., a camera, microphones, sensors) may be set up in an operating room 300 (as shown in FIG. 3) and synchronized with operating room data sources, such as a patient's electronic health record (EHR). As part of existing EHR workflows, key transitions in phases of the patient's care are documented within the intraoperative record, as shown in Table 1 in FIG. 6 depicting a particular anesthesia EHR. By synchronizing the EHR data within the digital recording, the EHR data-synchronized digital recording may be considered to be a multimodal surgical data comprising EHR data, video data, physiologic data, and audio data that are synchronized to a common timeline.

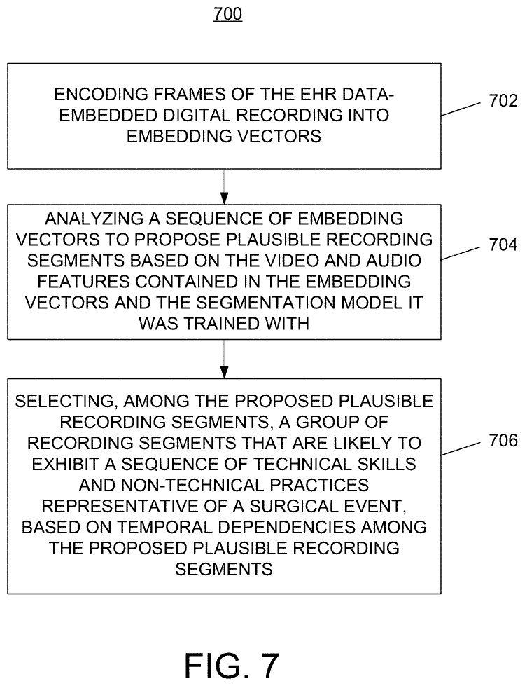

In some embodiments, the digital recording may be divided into temporal recording segments via the segmentation model (e.g., segmentation model 202). To do so, method 700 may be performed. As shown in FIG. 7, method 700 may generally encode frames of the EHR data-synchronized digital recording into embedding vectors, as shown in block 702, analyze a sequence of embedding vectors to propose plausible recording segments based on the values contained in the embedding vectors, as shown in block 704, and select, among the proposed segments, a group of recording segments that are likely to exhibit a sequence of technical skills and/or non-technical practices representative of a surgical phase, based on temporal dependencies among the proposed segments, as shown in block 706.

Turning back to FIG. 5, method 500 may then analyze the one or more segments to (i) characterize a plurality of independent features associated with a technical skill and/or a non-technical practice that are evident in the one or more segments and (ii) determine a higher-order pattern based upon analyzing a group of at least two of the plurality of independent features, via the video/understanding model (e.g., the video/audio understanding model 204), as shown in block 504. In an embodiment, a CNN may learn the video/audio understanding model 204. The CNN, which may include several convolutional layers and several fully connected layers, may analyze spatial, optical flow, and audio features of a digital recording represented by embedding vectors described above. FIG. 4A shows a recording segment 402 that is likely to exhibit a sequence of technical skills (e.g., scalpel usage) and non-technical practices (i.e., assistance from team members) representative of a surgical phase selected by the server 104. The server 104 may analyze the plot depicted in FIG. 4B to extract independent features (e.g., a mean velocity or other metric, such as the peak velocity of approximately 1.6 during transition of the scalpel, time duration of suturing) and determine a higher-order pattern (e.g., suturing efficiency) based upon analyzing a group of the independent features. Other features across different phases of an operation are illustrated in Table 2 as shown in FIG. 6.

Method 500 may then compare the higher-order pattern to ratings data (e.g., metrics associated with OSATS and/or NOTSS) associated to outcomes following one or more surgical procedures, as shown in block 506, and subsequently automatically generate a quality score based upon the comparing, as shown in block 508. As such, the quality score may be predictive of an assessment of the technical skill and/or non-technical practice. In some embodiments, quality scores may be binary (e.g., “good,” “bad”). In other embodiments, quality scores be provide additional details for several categories of a surgical phase. For example, a quality score for suturing may be (10, 10) for the categories “speed” and “suture placement” on a 10 point scale. In yet other embodiments, quality scores may be adapted to metrics associated with NOTSS, ANTS, PINTS, OSATS scoring systems.

The following additional considerations apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

The patent claims at the end of this patent application are not intended to be construed under 35 U.S.C. § 112(f) unless traditional means-plus-function language is expressly recited, such as “means for” or “step for” language being explicitly recited in the claim(s). The apparatus and methods described herein are directed to an improvement to computer functionality, and improve the functioning of conventional computers.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present). Herein, the term “set” may refer to any collection, list, bucket, etc. of items (including other sets) of one or more repeating or non-repeating items, whether sorted or not.

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium) or hardware. In hardware, the routines, etc., are tangible units capable of performing certain operations and may be configured or arranged in a certain manner. In example embodiments, one or more computer devices (e.g., a standalone, client or server computer device) or one or more modules of a computer device (e.g., a processor or a group of processors) may be configured by software (e.g., an application or application portion) as a module that operates to perform certain operations as described herein.

In various embodiments, a module may be implemented mechanically or electronically. For example, a module may comprise dedicated circuitry or logic that is permanently configured (e.g., as a special-purpose processor, such as a field programmable gate array (FPGA) or an application-specific integrated circuit (ASIC) to perform certain operations. A module may also comprise programmable logic or circuitry (e.g., as encompassed within a general-purpose processor or other programmable processor) that is temporarily configured by software to perform certain operations. It will be appreciated that the decision to implement a module mechanically, in dedicated and permanently configured circuitry, or in temporarily configured circuitry (e.g., configured by software) may be driven by cost and time considerations.

Accordingly, the term “module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which modules are temporarily configured (e.g., programmed), each of the modules need not be configured or instantiated at any one instance in time. For example, where the modules comprise a general-purpose processor configured using software, the general-purpose processor may be configured as respective different modules at different times. Software may accordingly configure a processor, for example, to constitute a particular module at one instance of time and to constitute a different module at a different instance of time.

Modules can provide information to, and receive information from, other modules. Accordingly, the described modules may be regarded as being communicatively coupled. Where multiple of such modules exist contemporaneously, communications may be achieved through signal transmission (e.g., over appropriate circuits and buses) that connect the modules. In embodiments in which multiple modules are configured or instantiated at different times, communications between such modules may be achieved, for example, through the storage and retrieval of information in memory structures to which the multiple modules have access. For example, one module may perform an operation and store the output of that operation in a memory product to which it is communicatively coupled. A further module may then, at a later time, access the memory product to retrieve and process the stored output. Modules may also initiate communications with input or output products, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented modules. The performance of certain of the operations may be distributed among the one or more processors, not only residing within a single machine, but deployed across a number of machines. In some example embodiments, the processor or processors may be located in a single location (e.g., within a building environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a building environment, an office environment, or a server farm). In other example embodiments, the one or more processors or processor-implemented modules may be distributed across a number of geographic locations.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Upon reading this disclosure, those of skill in the art will appreciate still additional alternative structural and functional designs for the methods and apparatus described herein through the principles disclosed herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed:

1. A computer-implemented method of characterizing and evaluating a surgical procedure, the method comprising:
receiving, by one or more processors, one or more segments of a digital recording, wherein the one or segments include video and/or audio data of a surgical procedure;
analyzing, by the one or more processors via a video/audio understanding model, the one or more segments to (i) characterize a plurality of independent features associated with a technical skill and/or a non-technical practice that are evident in the one or more segments and (ii) determine a higher-order pattern based upon analyzing a group of at least two of the plurality of independent features;
comparing, by the one or more processors, the higher-order pattern to ratings data associated to outcomes following one or more surgical procedures; and
automatically generating, by the one or more processors, a quality score based upon the comparing, wherein the quality score is predictive of an assessment of the technical skill and/or non-technical practice.

2. The computer-implemented method of claim 1, wherein the video/audio understanding model was trained by comparing the video and/or audio data to labeled data that identifies the plurality of independent features.

3. The computer-implemented method of claim 2, wherein the labeled data comprises at least one of human annotation data or electronic health record (EHR) data.

4. The computer-implemented method of claim 1, wherein the plurality of independent features associated with the technical skill correspond to economy of motion of a surgical tool or a hand of a medical professional.

5. The computer-implemented method of claim 4, wherein the higher-order pattern comprises suturing efficiency.

6. The computer-implemented method of claim 1, wherein the plurality of independent features associated with the non-technical practice correspond to volume or frequency of verbal cues.

7. The computer-implemented method of claim 1, wherein the video/audio understanding model was trained using at least one of support vector machines (SVMs), ensemble classifiers, or artificial neural networks (ANNs).

8. The computer-implemented method of claim 1, wherein the one or more segments were generated by dividing the digital recording via a learned segmentation model.

9. The computer-implemented method of claim 8, wherein the learned segmentation model was trained using at least one of support vector machines (SVMs), ensemble classifiers, or artificial neural networks (ANNs).

10. The computer-implemented method of claim 8, wherein the learned segmentation model is configured to:
encode frames of the digital recording into embedding vectors;
analyze a sequence of embedding vectors to propose plausible recording segments; and
select, among the proposed plausible recording segments, the one or more segments likely to exhibit a sequence of technical skills and/or non-technical practices representative of the surgical procedure, based on temporal dependencies among the proposed plausible recording segments.

11. A surgical procedure identification and rating device, comprising:
one or more processors; and
an application comprising a set of computer-executable instructions stored on one or more memories, wherein the set of computer-executable instructions, when executed by the one or more processors, cause the one or more processors to:
receive one or more segments of a digital recording, wherein the one or segments include video and/or audio data of a surgical procedure;
analyze, via a video/audio understanding model, the one or more segments to (i) characterize a plurality of independent features associated with a technical skill and/or a non-technical practice that are evident in the one or more segments and (ii) determine a higher-order pattern based upon analyzing a group of at least two of the plurality of independent features;
compare the higher-order pattern to ratings data associated to outcomes following one or more surgical procedures; and
automatically generate a quality score based upon the comparing, wherein the quality score is predictive of an assessment of the technical skill and/or non-technical practice.

12. The surgical procedure identification and rating device of claim 11, wherein the video/audio understanding model was trained by comparing the video and/or audio data to labeled data that identifies the plurality of independent features.

13. The surgical procedure identification and rating device of claim 12, wherein the labeled data comprises at least one of human annotation data or electronic health record (EHR) data.

14. The surgical procedure identification and rating device of claim 11, wherein the plurality of independent features associated with the technical skill correspond to economy of motion of a surgical tool or a hand of a medical professional.

15. The surgical procedure identification and rating device of claim 14, wherein the higher-order pattern comprises suturing efficiency.

16. The surgical procedure identification and rating device of claim 11, wherein the plurality of independent features associated with the non-technical practice correspond to volume or frequency of verbal cues.

17. The surgical procedure identification and rating device of claim 11, wherein the video/audio understanding model was trained using at least one of support vector machines (SVMs), ensemble classifiers, or artificial neural networks (ANNs).

18. The surgical procedure identification and rating device of claim 11, wherein the one or more segments were generated by dividing the digital recording via a learned segmentation model.

19. The surgical procedure identification and rating device of claim 18, wherein the learned segmentation model was trained using at least one of support vector machines (SVMs), ensemble classifiers, or artificial neural networks (ANNs).

20. The surgical procedure identification and rating device of claim 18, wherein the learned segmentation model is configured to:
encode frames of the digital recording into embedding vectors;
analyze a sequence of embedding vectors to propose plausible recording segments; and
select, among the proposed plausible recording segments, the one or more segments likely to exhibit a sequence of technical skills and/or non-technical practices representative of the surgical procedure, based on temporal dependencies among the proposed plausible recording segments.
